(19) United States
(12) Patent Application Publication
(10) Pub. No.: US 2013/0012763 A1
(43) Pub. Date: Jan. 10, 2013

(54) System and a Method for Controlling an Environmental Physical Characteristic,

(75) Inventor: Stijn De Waele, Ossining, NY (US)

(73) Assignee: KONINKLIJKE PHILIPS ELECTRONICS, N.V., Eindhoven (NL)

(21) Appl. No.: 13/636,005

(22) PCT Filed: Mar. 16, 2011
(86) PCT No.: PCT/IB2011/051094

§ 371 Date: Sep. 19, 2012

(30) Foreign Application Priority Data
Mar. 25, 2010 (EP) 10157701.3

(51) Int. Cl. A61M 21/02

(52) U.S. Cl. 600/28; 600/26; 600/27

(57) ABSTRACT

A system (100) and a method for controlling an environmental physical characteristic in an environment of a person (105), a color and intensity tunable lamp, an electronic device and a computer program product are provided. The system (100) comprises an environment influencing means (102), a sensor (104), an input means (134) and a controller (130). The environment influencing means (102) changes the environmental physical characteristic in response to a control signal (112) indicating a desired influencing of the environmental physical characteristic. The sensor (104) obtains a sensor signal (122) representing a physiological condition of the person (105). The input means (134) obtains a user setting signal (132) representing a user setting with respect to the environmental physical characteristic. The controller (130) receives the sensor signal (122) and the user setting signal (132) and generates the control signal (112) by applying an original function (114) to the sensor signal (122). The controller (130) defines an adapted function in response to receiving the user setting signal (132), wherein the adapted function provides the control signal(112) which, if applied to the sensor signal (122) occurring when the user setting signal(132) was received, causes the environmental physical characteristic to become closer to the user setting than when the original function (114) is applied to the sensor signal occurring when the user setting signal (132) was received. The controller (130) subsequently applies the adapted function. Finally, the controller (130) gradually changes the applied adapted function towards another function.

### FIELD OF THE INVENTION

The invention relates to the field of systems for controlling an environmental physical characteristic in an environment of a person.

### BACKGROUND OF THE INVENTION

Colored lamps are often used to change the characteristics of environmental light in the environment of a person to influence the psychological state or physiological state of the person. For example, a sleeping person is influenced by the intensity of light such that his sleep intensifies or such that the person awakes. The color of the light may determine, for example, the mood of the person. Today, lamps are available which emit a specific color in response to a user input. A user may provide a specific input which matches his mood. However, if the mood of the user changes, the color of the emitted light remains the same and the emitted color does not match the changed mood of the user.

Published patent application EP2100556 discloses an apparatus and a method for modifying a psychophysiological state of a person. On the basis of detected psychophysiological signals, an estimate of the psychophysiological state of the person is obtained. Stimuli are provided to the person on the basis of the estimate. The provided stimuli influence the psychophysiological state of the person. The stimuli are, for example, audio, light, video, haptic or scent stimuli. The apparatus and the method utilize pre-programmed knowledge and/or a pre-programmed algorithm to determine the stimuli that have to be provided to the person such that the stimuli match with the psychophysiological state of the person. The method and the apparatus do not have an interface which may be used by the user to switch to a specific set of user preferred stimulus parameters, for example, to set the environmental light to a specific intensity or to a specific color.

In general, users prefer to have the possibility to modify the current behavior of the system that controls environmental parameters through stimuli by providing user input. The supplied user input most probably relates to stimuli which match the mood of the user at the time of supplying the user input. The supplied user input may also relate to functional requirements, like, for example, increasing the room temperature to increase the body temperature, or increasing the light intensity such that the user is able to read a document more easily. Thus, the supplied user input relates to a temporary situation in which the user requires a specific behavior of the system. However, in general, the system is meant to control stimuli in accordance with a certain physical model or statistical model that is based on a number of experiments.

### SUMMARY OF THE INVENTION

It is an object of the invention to provide a more user friendly system for controlling physical characteristics relating to an environment of a person.

A first aspect of the invention provides a system for controlling an environmental physical characteristic in an environment of a person as claimed in claim 1. A second aspect of the invention provides a lamp as claimed in claim 12. A third aspect of the invention provides an electronic device as claimed in claim 13. A fourth aspect of the invention provides a method of controlling an environmental physical characteristic in an environment of a person as claimed in claim 14. A fifth aspect of the invention provides a computer program product as claimed in claim 15. Advantageous embodiments are defined in the dependent claims.

A system for controlling an environmental physical characteristic in an environment of a person in accordance with the first aspect of the invention comprises an environment influencing means, a sensor, an input means and a controller. The environment influencing means changes the environmental physical characteristic in response to a control signal which indicates a desired influencing of the environmental physical characteristic. The sensor obtains a sensor signal which represents a physiological condition of the person. The input means obtains a user setting signal which represents a user setting with respect to the environmental physical characteristic. The controller receives the sensor signal and the user setting signal and generates the control signal by applying an original function to the sensor signal. In response to receiving a user setting signal the controller defines an adapted function. The adapted function provides the control signal which, if applied to the sensor signal occurring when the user setting was received, causes the environmental physical characteristic to become closer to the user setting than when the original function is applied to the sensor signal occurring when the user setting was received. Subsequently, the controller applies the adapted function. Thereafter the applied adapted function is gradually changed towards another function.

A short time interval after the moment that the user inputs the user setting, the environment influencing means changes the physical characteristic of the environment such that the physical environmental characteristic becomes closer to the user setting, because the controller applies the adapted function which provides a control signal that is closer to the user setting when the adapted function is applied to the sensor signal occurring when the user setting was received. The user experiences the system as a more user-friendly system because the system responds as soon as possible to the received user setting and responds such that the influencing of the physical environmental characteristic relates to the user setting.

Further, the system gradually changes the applied adapted function in the time period after receiving the user setting, because the received user setting was most relevant for the user at the particular instant when the user setting was provided by the user. The change allows, for example, changing the function towards a function of which is known a priori that users experience, in general, the response of the system as a pleasant response which matches with their physiological state.

The provided user setting may be random to some extent, which means that the provided user setting does not follow a specific pattern in relation to the physiological state of the person. If the same user, or another user, uses the system later on, he does not want to be confronted with behavior of the system that is based on the more or less random input setting provided. And thus, the single user or other users are only bothered with the user setting to a certain extent, namely, the gradually changed function still depends to a certain extent on the applied adapted function.

In an embodiment, the adapted function provides the control signal which, if applied to the sensor signal occurring when the user setting was received, causes the environmental physical characteristic to become substantially equal to the user setting.

The system is experienced as more user-friendly if the influencing of the environmental physical characteristic is in accordance with the provided user setting.

In another embodiment, the controller applied a previous function before the particular moment of receiving the user setting signal, and the controller is configured for combining a further function with a limited bandwidth with the previous function to define the adapted function. The limited bandwidth is a predefined range in which the further function provides a value substantially different from zero and the limited bandwidth is arranged around the value of the sensor signal occurring when the user setting signal (132) was received.

In other words, while defining the function in response to receiving the user setting signal, the controller uses the function that was applied before a particular moment of receiving the user setting signal and uses a function with a limited bandwidth, the bandwidth being arranged around the value of the sensor at the particular moment. Combining the previous function and the function with the limited bandwidth may comprise one or more mathematical operations, like, for example, adding the function with the limited bandwidth to the previous function, subtracting the function with the limited bandwidth from the previous function, or generating a weighted average of both functions. It is to be noted that combining these functions has to be performed such that the adapted function still fulfills the condition that the adapted function provides the control signal comprising the user setting if the sensor signal is substantially equal to the sensor signal of the particular instant at which the user setting signal was received.

It is advantageous to use the function that was applied just before the particular moment, because the previous function is most probably a function that well represents, in general, a desirable model-based relation between the physiological conditions of persons and the environmental physical characteristic. Thus, the adapted function is to some extent influenced by the user setting and to a large extent still based on the model.

Further, combining the use of the function with a limited bandwidth is advantageous because the adapted function differs only from the previous function in the bandwidth around the value of the sensor signal at the particular instant at which the user setting was received. Thus, if the adapted function is applied, and the physiological state of the person changes significantly, the adapted function behaves according to the previous function. A significant change of the physiological state is defined by a change such that the value of the sensor signal changes to a value outside the bandwidth. As discussed, the previous function incorporates a specific model which defines the relation between the sensor signal and the control signal. It is advantageous to return to the model-based behavior of the system if the physiological state changes a lot.

In another embodiment, the controller applied a previous function before the particular moment of receiving the user setting signal, and the controller is configured for combining a symmetrical function with the previous function to define the adapted function, wherein the symmetrical function is arranged around the value of the sensor signal at the particular instant at which the user setting was received.

Combining the symmetrical function with the previous function is advantageous because the system which applies the adapted function shows similar behavior whether the value of the sensor signal is increasing or decreasing. This is experienced as user-friendly behavior. Further, the symmetrical function may be selected such that it has a limited bandwidth.

In an embodiment the further function is based on a Gaussian function.

The Gaussian function is a symmetrical function centered around a mean value. The mean of the Gaussian function may be chosen such that the function is symmetrical with respect to the value of the sensor signal at the particular instant at which the user setting was received. A further characteristic of a Gaussian function is that, when the input variable to the function is outside a certain interval around the mean value, the function provides a value substantially equal to zero. Thus, if the Gaussian function is shifted and added to, or subtracted from, the previous function, the behavior of the adapted function at input values far enough from the value of the sensor signal at the particular instant is substantially equal to that of the previous function. This is advantageous, because the user setting represents only the combination of “user setting for the environmental physical characteristic” and “the value of the sensor signal at the particular instant”, and does not have a relation with sensor signals far enough from the value of the sensor signal at the particular instant.

It is to be noted that the function with a limited bandwidth is based on a Gaussian function, which means that it may be a shifted and/or scaled Gaussian function.

In an embodiment, the controller defines the adapted function such that, if the sensor signal varies within a predefined variability interval around the value of the sensor signal of the particular moment, the control signal remains within a predefined deviation interval.

If a user provides a user setting, and the system influences the environmental physical characteristic according to the user setting, the user does not want that the environmental physical characteristic changes much if his physiological condition remains within a specific variability interval. This behavior of the system may be obtained by selecting a symmetrical function that remains reasonably flat in a range around the value of the sensor signal at the particular moment, for example, by selecting a normal distribution with a large enough variance (a) such that the adapted function fulfills the condition.

The sensor measures a physiological condition of the person and the measured characteristic of the person may vary within an interval, while the physiological state is experienced by the person as a stable condition. For example, a heartbeat of a person varies within a specific interval while the person is still in the same physiological state. The variation of the measured characteristic may form the basis for the variability interval.

In another embodiment, the controller starts the gradual change of the applied function only after receiving the sensor signal with a value outside a predefined range around the value of the sensor signal occurring when the user setting signal (132) was received.

In this embodiment, as long as the user is in a physiological condition which, allowing for a predefined error margin, is substantially equal to the physiological condition at the particular instant, the system of the embodiment does not gradually change the applied adapted function. Thus, the influencing of the environmental physical characteristic remains in accordance with the user setting. Users experience this behavior of the system as user-friendly, because it seems that the system listens to the settings of the user. If, however, the physiological state of the user changes more than a predefined variability margin, the system starts to gradually change the applied adapted function.

As discussed in the previous embodiment, the sensor may provide values within a variability interval while the person is in the same physiological condition. This embodiment prevents that the control signal changes while the person is still in the same physiological condition.

In an embodiment, the controller stores a standard predefined function. The gradual change of the applied function is towards the standard predefined function.

Thus, in other words, said another function is the standard predefined function. The standard predefined function may be a function that is based on a large number of field tests, of which the results are combined in a statistical model, or the standard predefined function may be based on a physical model. Thus, the standard predefined function represents, except at the moments that a user setting is received, the desired behavior of the system which is in general experienced by users as a pleasant or desirable response of the system. Therefore, it is advantageous to change the applied adapted function towards the standard predefined function, because it is known a priori that most users of the system experience that response of the system according to the standard predefined function as a desirable response.

In another embodiment, the controller applied a previous function before the particular instant of receiving the user setting signal. The controller further defines, after defining the adapted function, said another function by combining the adapted function and the previous function.

In other words, said another function is generated such that it is partly based on the function that was applied by the controller before the particular instant of receiving the user setting signal, and partly based on the user setting. Thus, said another function is a function which takes into account the user setting, but only to a certain degree, and which is based on the history before the particular moment. If at several instants the user provides his setting, said another function iteratively evolves towards a function that is based on the function before any user setting was received and the history of received user settings. This is user-friendly because the system adapts to the user. Especially, if one user provides a consistent scheme of user settings, said another function evolves towards a function that clearly includes this consistent scheme.

Combining the previous function and the adapted function may comprise one or more mathematical operations, like for example, adding the adapted function to the previous function, or subtracting the adapted function from the previous function.

In a further embodiment, said another function is a weighted average of the previous function and the adapted function.

By creating a weighted average, said another function is a statistical representation of the past, of which information is provided via the previous function, and the present user setting, of which information is provided via the adapted function. A statistical representation is advantageous because a system applying such a statistical representation is most probably experienced by most users as a pleasant system.

In an embodiment, the environmental physical characteristic comprises at least one of the following: characteristics of light of the environment, characteristics of sound in the environment, temperature, humidity, characteristics or movements of objects or parts of objects in the environment, characteristics or movements of objects with which the person is in physical contact or a scent of the environment.

The environmental physical characteristic may be any physical characteristic of the environment of the person which may be sensed by at least one of the sense organs of a human being, such as the vision, auditory, touch, proprioceptive and olfactory sensory organs.

The characteristics or movements of objects with which the person is in physical contact relates to physical characteristics which may be felt by the person at the contact area between the object and the person, for example, the temperature of the object, or movements of the object. Characteristics of objects with which the person is not in direct contact may also be sensed by the person, for example, by means of visual detection. Such a characteristic may be that an object has a color, or moves according to a pattern or at a specific speed.

In an embodiment, the environment influencing means comprises at least one of the following: a light emitting device, a controllable light filter, a controllable curtain, a sound emitting device, a sound cancellation device, a heating device, a cooling device, a humidifier, an actuator, an odouriser or a haptic device.

In specific embodiments of the environment influencing means, the physical characteristic of the environment may be changed instantaneously in response to a received control signal, for example, by immediately providing light of a specific color according to the control signal. In other embodiments of the environment influencing means, the change of the physical characteristic of the environment is performed gradually, which is, for example, the case when the control signal indicates a temperature which requires a temperature change. Depending on the heating or cooling capacity of the heating or cooling device and depending on the thermal capacity of the environment, only after a time period the temperature will be substantially equal to the level which is indicated by the control signal.

It is to be noted that, for example, an inflatable mattress with a controllable air pump may also be an environment influencing means. In a system comprising the inflatable mattress, the sensor may obtain information related to the physiological condition of a sleeping person, and depending on the condition air is pumped into or released from the mattress in order to obtain a softness of the mattress that matches the physiological condition of the sleeping person. The user-setting is, for example, provided to the system just before the person goes to sleep to control the softness of the mattress during the beginning of the sleeping period.

In a further embodiment, the sensor signal comprises information relating to at least one of the following physiological conditions of the person: skin temperature, core body temperature, skin conductivity, blood pressure, heart rate, heart rate variability, movements of the person, respiration frequency, respiration frequency variability, EEG activity, eye activity, characteristics of sound directly related to the person or ECG information.

The information relating to the physiological condition of the person is obtained by the sensor and is sent to the controller by means of the sensor signal. The information may be any type of information relating to the person. The sensor may be in direct contact with the person, for example, to measure ECG or EEG activity, core body temperature or skin conductivity, or may be a sensor which is located in the neighborhood of the person and detects characteristics of the person. Such a sensor may be a microphone which detects sound made by the person, or may be an infrared sensor which detects the skin temperature, or may be a camera and a video processing system which obtains information related to movements of the person, for example, the respiratory movement.

According to the second aspect of the invention, a color and intensity tunable lamp is provided which comprises the system according to the first aspect of the invention.

According to the third aspect of the invention, an electronic device is provided comprising the system according to the first aspect of the invention.

According to the fourth aspect of the invention, a method of controlling an environmental physical characteristic in an environment of a person is provided. The method comprises the step of obtaining a sensor signal by a sensor. The sensor signal represents a physiological condition of the person. The sensor signal is provided to a controller. In a further step the controller generates a control signal by applying an original function to the sensor signal. The control signal indicates a desired influencing of the environmental physical characteristic. The control signal is provided to an environment influencing means. The environmental physical characteristic is changed by the environmental influencing means in response to the control signal. In a further step a user setting signal is obtained by an input means. The user setting signal represents a user setting with respect to the environmental physical characteristic. The user setting signal is provided to the controller. In a further step the controller defines an adapted function in response to receiving the user setting signal. The adapted function provides the control signal which, if applied to the sensor signal occurring when the user setting was received, causes the environmental physical characteristic to become closer to the user setting than when the original function is applied to the sensor signal occurring when the user setting signal was received. Subsequently, the adapted function is applied in the controller to generate the control signal. In a further step the controller gradually changes the applied adapted function towards another function.

According to the fifth aspect of the invention, a computer program product is provided which comprises instructions for causing a processor system to perform the method according to the fourth aspect of the invention.

The lamp according to the second aspect of the invention, the electronic device according to the third aspect of the invention, the method according to the fourth aspect of the invention and the computer program product according to the fifth aspect of the invention provide the same benefits as the system according to the first aspect of the invention and have similar embodiments with similar effects as the corresponding embodiments of the system.

These and other aspects of the invention are apparent from and will be elucidated with reference to the embodiments described hereinafter.

It will be appreciated by those skilled in the art that two or more of the above-mentioned embodiments, implementations, and/or aspects of the invention may be combined in any way deemed useful.

Modifications and variations of the system, the method, and/or of the computer program product, which correspond to the described modifications and variations of the system, can be carried out by a person skilled in the art on the basis of the present description. The sensor signal may comprise more dimensional information, for example, representing more than physiological parameters of the person, and the control signal may comprise more dimensional information, for example, the desired influencing of a plurality of environmental physical characteristics. The function may be a function that maps a vector to another vector, wherein each one of the vectors represents multidimensional information.

### BRIEF DESCRIPTION OF THE DRAWINGS

In the drawings:

FIG. 1 schematically shows the system in accordance with the first aspect of the invention,

FIG. 2 schematically shows the combining of a function with a symmetrical function, and shows the gradually change of the function,

FIG. 3 schematically shows the combining of a function with a normal distribution, and shows another function which is a weighted average of functions,

FIG. 4 schematically shows the evolution of function when a user feedback was provided twice within a relative short time interval,

FIG. 5a schematically shows a lamp according to the second aspect of the invention,

FIG. 5b schematically shows an electronic device according to the third aspect of the invention, and

FIG. 6 shows a flow diagram of the method according to the fourth aspect of the invention.

It should be noted that items denoted by the same reference numerals in different Figures have the same structural features and the same functions, or are the same signals. Where the function and/or structure of such an item have been explained, there is no need for repeated explanation thereof in the detailed description.

The figures are purely diagrammatic and not drawn to scale. Particularly for clarity, some dimensions are exaggerated strongly.

### DETAILED DESCRIPTION OF THE EMBODIMENTS

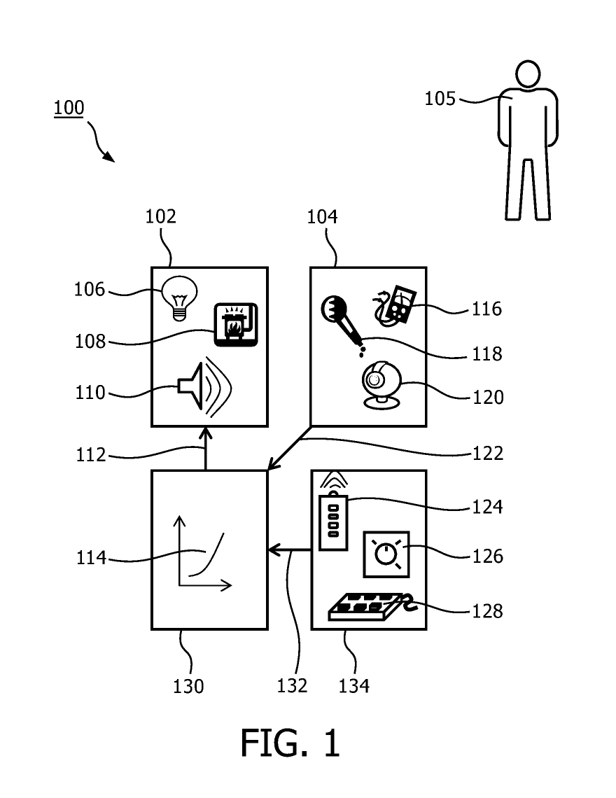

A first embodiment is shown in FIG. 1. A system 100 controls an environmental physical characteristic in an environment of a person 105. The system comprises an environment influencing means 102, a sensor 104, an input means 134 and a controller 130. The environment influencing means 102 changes the environmental physical characteristic in response to a control signal 112. The control signal 112 indicates a desired influencing of the environmental characteristic. The sensor 104 obtains a sensor signal 122 which represents a physiological condition of the person 105. The input means obtains a user setting signal 132 which represents a user setting with respect to the environmental physical characteristic. The controller 130 receives the sensor signal 122 and the user setting signal 132. The controller 130 generates the control signal 112 by applying a function 114 to the sensor signal 122. In response to receiving the user setting signal 132 the controller 130 defines the adapted function such that the adapted function provides the control signal comprising the user setting if the sensor signal 122 is substantially equal to the sensor signal of a particular instant at which the user setting signal 132 was received. The adapted function is applied by the controller 130. After the particular instant at which the user setting signal 132 was received, the controller 130 gradually changes the applied adapted function.

As seen in FIG. 1, the environment influencing means may be any means that is capable of changing the environmental physical characteristic in response to receiving a control signal 112. If the environmental physical characteristic is, for example, light, a lamp 106 may be used. If the temperature has to be changed, a heater 108 may be used, or in another embodiment the environmental physical characteristic is sound which may be influenced by a speaker 110.

Different sensors may be used to obtain the sensor signal 122 representing a physiological condition of the person 105. A microphone 118 may be used to detect sounds related to person 105, a webcam 120 may detect movements of the person 105, or, for example, eye activity of the person 105. A conductivity and temperature sensor 116 may have pads which are in contact with the skin of the person 105 and may generate a signal comprising a skin conductivity value and a value representing the skin temperature of the person 105.

The input means 134 may be any means which allows the person 105 to provide input which relates to the environmental physical characteristic. The controller 130 interprets this input as a user setting and defines the adapted function accordingly. Examples of input means are a remote controller 124, a keyboard 128 or a kind of thermostat 126 which allows the person 126 to indicate which environmental temperature is preferred.

As discussed in the examples of the environment influencing means 102, the sensor 104 and the input means 134, the environmental physical characteristic may be any physical characteristic which relates to the environment of the person 105. A non-exhaustive list of examples comprises: characteristics of light of the environment, characteristics of sound in the environment, temperature, humidity, characteristics or movements of objects or parts of objects in the environment, characteristics or movements of objects with which the person is in physical contact or a scent of the environment.

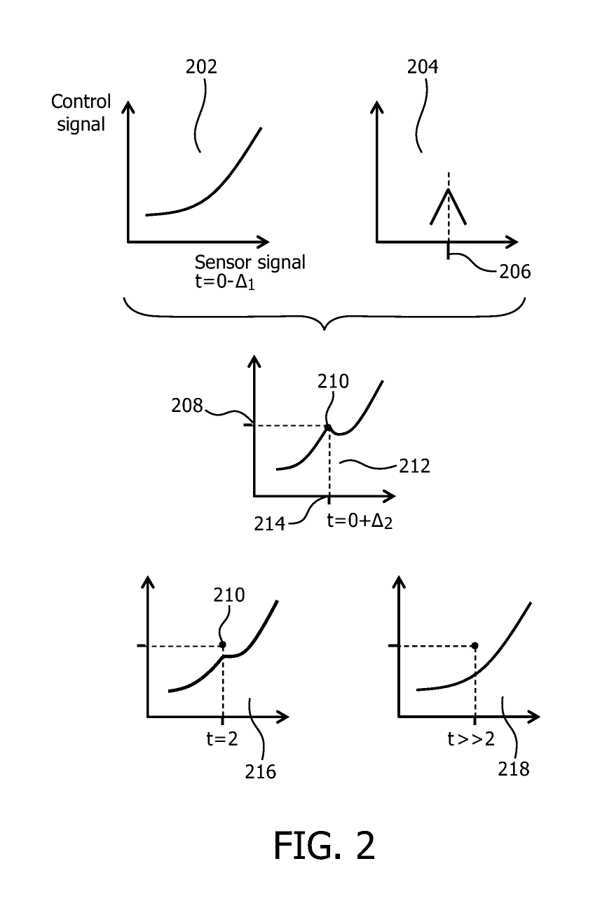

FIG. 2 schematically shows the defining of the function and the gradual changing of the function towards said another function. The previous function 202 shows the relation between the value of the sensor signal (along the x-axis) and the value of the control signal (along the y-axis). The previous function 202 is used by the controller just before the particular instant at which the user-setting signal was received by the controller. In FIG. 2, the particular instant at which the user-setting signal was received by the controller is defined as t=0, and thus the previous function is of an instant t=0−Δ0₁, wherein Δ₁ is a short period of time. The function 204 is a symmetrical function, which is symmetrical with respect to the value 206 of the sensor signal at the particular instant t=0.

The controller defines an adapted function 212 in response to receiving the user setting signal at t=0. The adapted function 212 is defined by combining the previous function 202 and the symmetrical function 204 by adding a scaled version of the symmetrical function 204 to the previous function 202. After a short time interval, indicated with the value Δ₂, at instant t=0+Δ₂, the generation of the adapted function 212 is finished and the adapted function 212 is applied by the controller to generate the control signal. The adapted function 212 has to fulfill a specific condition, namely, the point 210 has to be incorporated in the adapted function 212. Point 210 is defined by an x-value 214 which is substantially equal to the value of the sensor signal at the particular instant t=0, and an y-value 208, which is substantially equal to the received user setting with respect to the environmental physical character. Thus, as long as the controller applies the adapted function 212, the controller will generate a control signal comprising the user setting when the sensor signal is equal to the value of the sensor signal at the particular instant t=0.

However, after instant t=0+Δ₂, the controller gradually changes the applied adapted function 212 towards another function 218. In the example of FIG. 2, said another function 218 is equal to the previous function 202. The function 216 is the function which is applied by the controller after some time of gradually changing the applied adapted function 212, for example, at t=2. The Figure shows that the line of the function 216 does not go through point 210, which is the point that represents the user setting. It is also shown that the line of the function 216 still tends towards point 210 when the sensor signal is in close proximity to the value 214 of the sensor signal at the particular instant t=0. The gradual change is relatively slow such that the function applied in the controller becomes equal to said another function 218 long after the instant t=2. It is to be noted that the values of the time variable in the examples are presented without expressing a specific unit. In a practical example of influencing the light intensity, it may take up to 3 hours before the gradual change to another function is finalized. Thus, in the example of FIG. 2, t=2 may mean t is two quarters of an hour after t=0, and t>>2 means, for example, t=12. However, in other embodiments, depending on the specific environmental physical characteristic, the gradual change may be faster or slower.

In an embodiment, the previous function 202 may be a standard predefined function which is stored in the controller as a pre-programmed function. This function may be based on several field tests and, thus, represents a statistical average of functions which, when used in a system according to the first aspect of the invention, results in behavior of the system which is defined by users as convenient and/or desirable behavior. The standard predefined function may also be based on a physical model which represents a desirable relation between the sensor signal and the control signal. In this embodiment, said another function 218 is the standard predefined function. Thus, some time period after receiving the user setting signal, the controller changes the applied adapted function 212 toward a function which was used by the controller when the system was used for the first time.

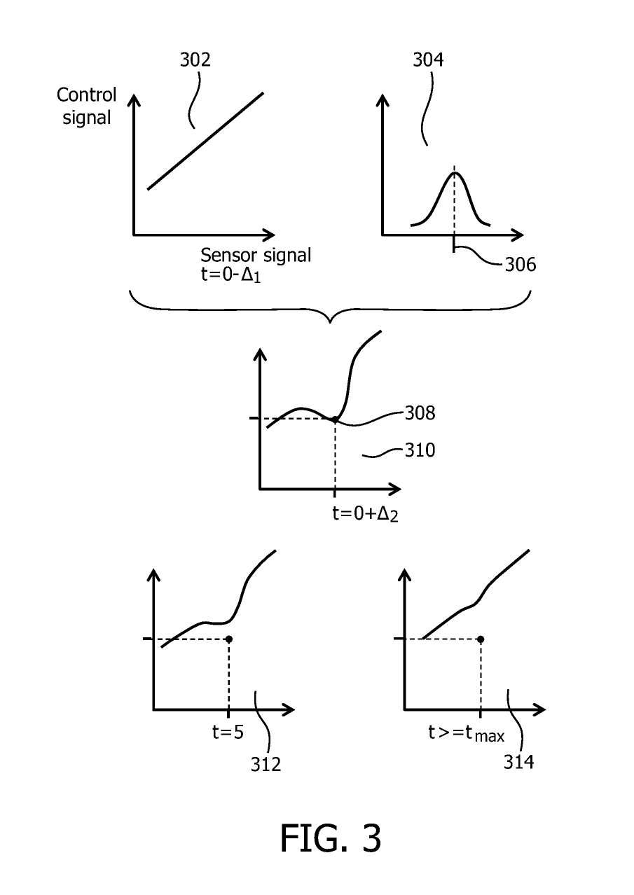

FIG. 3 illustrates another embodiment of the invention with a sequence of functions. At the moment t=0−Δ₁, a moment just before the particular instant t=0, the previous function 302 is applied by the controller. At the particular instant t=0, the value of the sensor signal substantially equals the value 306. A normal distribution 304, which is a Gaussian function, with a mean value 306 is combined with the previous function 302. The mean value 306 is the value of the sensor signal at the particular instant t=0, which is the instant at which the user setting signal was received by the controller. The normal distribution has a particular variance which will be discussed later.

At the particular moment t=0, the controller defines an adapted function 310 in accordance with the user setting signal. The previous function 302 and the normal distribution 304 are combined such that at t=0+Δ₂ the adapted function 310 is obtained. In the example of FIG. 3, a scaled version of the normal distribution 304 is subtracted from the previous function 302 such that point 308 is on the adapted function 310. At point 308 the adapted function provides a control signal comprising the user setting if the sensor signal equals the value of the sensor signal that was received at the particular moment t=0. After defining the adapted function 310, the controller applies the adapted function 310 to generate the control signal.

When the adapted function 310 is obtained by the controller, the controller obtains said another function 314 as well. Said another function 314 is a weighted average of the previous function 302 and the adapted function 310. In the example, the weight of the previous function 302 is 12 and the weight of the adapted function 310 is 1. By obtaining such another function 314, which is a weighted average, the influence of receiving a user setting signal is still present in said another function 314, while at the same moment said another function 314 has almost the same characteristic as the previous function 302.

In the example of FIG. 3, the controller starts changing gradually the applied adapted function 310 towards said another function 314 after t=0+Δ₂. The gradual change is limited to a time interval which is not longer than tₘₐₓ. Thus, as seen in FIG. 3, after some time the controller applies function 312, and finally, from t=tₘₐₓ onwards, said another function 314 is applied by the controller. If the physical environmental characteristic is, for example, the temperature of a room tₘₐₓ is in a practical embodiment the time period of 10 hours, then, in a practical embodiment the gradual change towards said another function is finalized after 10 hours.

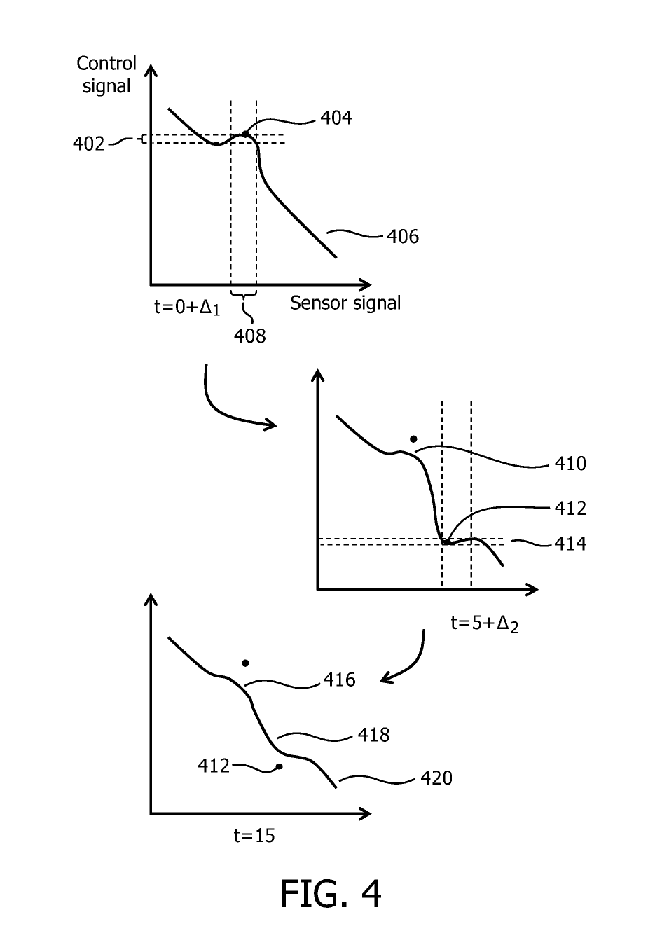

In FIG. 4, another example of an adapted function 406 is shown. At the particular instant t=0, a user setting was received which is incorporated in the first adapted function 406 at point 404 at instant t=0+Δ₁. The first adapted function 406 is, for example, the result of combining a previous function with a normal distribution. The variance of the normal distribution is chosen such that the first adapted function 406 has, when applied by the controller, the subsequent characteristic: if the value of the received sensor signal, which is received by the controller, varies within a predefined variability interval 408, the value of the generated control signal varies within a predefined deviation interval 402. In the example of FIG. 4, the predefined variability interval 408 is much smaller than the predefined deviation interval 402. Thus, the control signal remains close to the user setting, meaning that the value of the control signal varies only within the relatively small predefined deviation interval 402, while the sensor signal varies (a bit) more, meaning that the value of the sensor signal may vary within the larger predefined variability interval 408.

It is to be noted that the variability interval 408 may be related to the normal variation of the physiological characteristic that is sensed by the sensor. For example, the heartbeat of a person always varies to some extent around a mean value, and as such it is advantageous to keep the value of the control signal within the predefined deviation interval 402 if the value of the sensor value varies within the variability interval 408.

In the example of FIG. 4, at an instant t=5 another user setting was provided by the user. The controller used the gradually changed first adapted function which was used by the controller just before the instant t=5 and combined it with a normal distribution function to obtain at t=5+Δ₂ the shown second adapted function 414. The gradual changing of the first adapted function 406 may be observed in the second adapted function 414 because extrusion 410 protrudes less in the direction of point 404 than it does in the first adapted function 406. After defining the second adapted function 414, the controller applies the second adapted function 414 for generating the control signal.

The gradual change continues after t=5+Δ₂, as seen at the bottom of FIG. 4. For example, at t=15, the gradually changed second adapted function 420 is applied by the controller. Bump 416 protrudes less compared to extrusion 410, and extrusion 418 protrudes less in the direction of point 412. As seen in the gradually changed second adapted function 420, the influence of twice applying a user setting is present in the gradually changed second adapted function 420, however, the influence of the received user settings is reduced gradually.

In another embodiment, the gradual change does not start immediately after the instant t=0+Δ₁, but only after receiving for the first time, after the instant t=0, a sensor signal that is outside a predefined interval 408 around the value of the sensor signal at the instant t=0. Thus, as long as the person is in a physiological condition which is within the predefined physiological condition range at the moment that the user setting was applied by the person, the value of the control signal generated by the controller will be within the range 402, and, thus, the response of the system during such a period of time is about the same as the user setting which was provided by the person.

The subsequent embodiment is discussed with mathematical examples. The controller applies a function m:

c=m(x, t) (1)

wherein c is the vector representing the control signal, x is the vector representing the sensor signal and t is a variable representing time. By using a vector, it is indicated that the control signal and/or the sensor signal may be multi-dimensional signals, meaning that the environment influencing means may influence one or more physical environmental characteristics and meaning that the sensor senses one or more physiological characteristics of the person.

At the moment in time that the system in accordance with the first aspect of the invention is used for the first time, indicated with the particular instant t=0, a pre-programmed function m₀(x) is applied by the controller. Up to a particular instant at which the user provides a user setting to the system, the pre-programmed function m₀ is applied.

The particular instant at which the user provides a user setting is indicated with t=t₀. The user setting signal is represented by a user setting vector c*. At the particular instant the user is in a specific physiological state represented by a vector x*, which is the vector with the value(s) of the sensor signal at t₀. In response to receiving the user setting signal, the controller has to define a function which respects the received user setting, which may be expressed by the condition:

m(x*,t₀)=c* (2)

A further condition may be that said another function, which is applied by the controller a long period of time after t₀, is a weighted average of the pre-programmed function m₀(x) and a deviation of the user setting vector c* from the pre-programmed function in an area around x* (the physiological state of the user at t₀):

m(x, t>>t₀)=m_{inf}(x)=w(c*−m₀(x))G(x−x*)+(1−w)m₀(x) (3)

wherein G is centered around the null vector 0 and G(0)=1, and wherein the weight w is determined by a statistic reliability of the pre-programmed function m₀(x). If m₀(x) is based on n=100 previous experiments, w=1/n= 1/100 is used.

The following time-dependent function m₂ meets the conditions (2) and (3):

$${m}_{2} ( x , t ) = {e}^{- \frac{t - {t}_{0}}{τ}} ( {c}^{*} - {m}_{inf} ( x ) ) err ( \frac{\vert x - {x}^{*} \vert}{σ} ) + {m}_{inf} ( x ) \qquad ( 4 )$$

with said another function m_{inf}(x):

$${m}_{inf} ( x ) = w ( {c}^{*} - {m}_{0} ( x ) ) err ( \frac{\vert x - {x}^{*} \vert}{σ} ) + ( 1 - w ) {m}_{0} ( x ) \qquad ( 5 )$$

wherein err( . . . ) is a normalized error function or Gaussian function with variance 1 and τ is a parameter that controls the rate at which the function converges to a weighted average represented by said another function m_{inf}(x).

Thus, the function m₂(x, t₀) is the adapted function defined by the controller in response to receiving the user setting signal, and m₂(x, t) is the gradually changed, applied adapted function after the instant t=t₀.

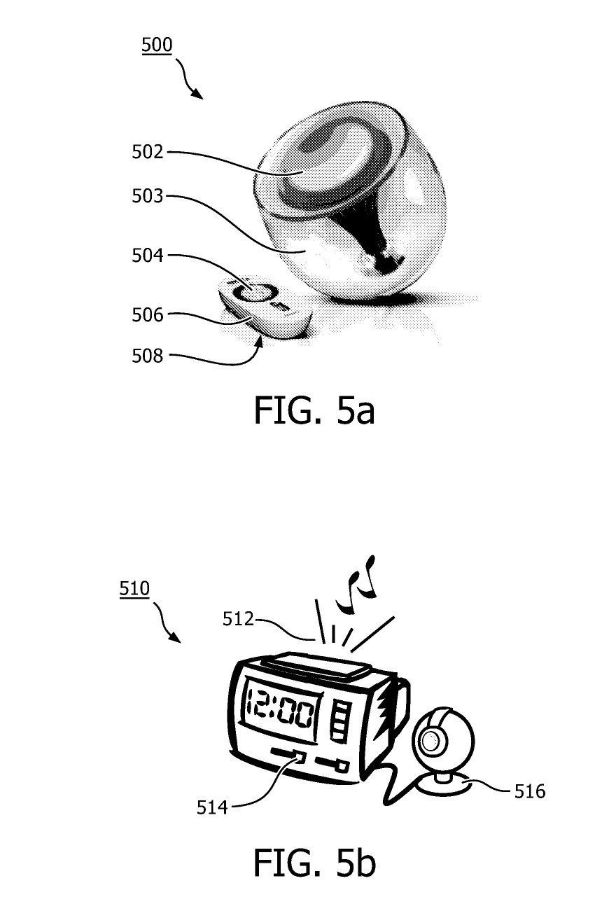

In FIG. 5a, a color and intensity tunable lamp 500 is presented according to the second aspect of the invention. The lamp 500 comprises the system according to the first aspect of the invention. Light emitter 502 is capable of emitting light of different colors at different intensities into the environment of a person. The remote controller 506 has two functions in the system. When the remote controller 506 is touched by the person, the bottom 508 of the remote controller 506 measures by means of two electrodes the heart beat of the person. The number of heart beats per minute is provided to a controller. The remote controller 506 further comprises a so-termed color wheel which may be used by the person to provide his user setting with respect to the color of the light which is emitted by the light emitter 502. The color wheel provides the user setting to the controller. The controller may be arranged in the remote controller 506 or in the luminaire 503. If the controller is positioned within the remote controller 506, the control signal is transmitted from the remote controller 506 to the light emitter 502. If the controller is positioned within the luminaire, a user setting signal and a sensor signal are transmitted from the remote controller 506 to the controller. The controller operates in accordance with previous embodiments. Another selector on the remote controller 506 may be used by the user to provide his preference with respect to the light intensity emitted by the light emitter 502.

FIG. 5b shows an electronic device 510, being an alarm clock with an integrated radio. The alarm clock 510 may be switched on by a person who goes to bed to listen to music 512. The alarm clock 510 further comprises a video camera 516 to observe the person who is going to sleep. The images of the video camera are analyzed to detect the physiological state of the person with respect to a physiological condition of the person related to “being awake or sleeping”. The video analyzer generates a sensor signal indicating the sleeping-state of the person. The controller of the alarm clock 510 applies a standard function to generate a control signal which indicates the required volume level of the music 512 played. The standard function represents the subsequent behavior of the alarm clock: if the person is in a sleeping state, the volume is reduced to a low level; if the person is awake, the volume is controlled to have an average intensity level. Detecting the physiological state of the person is done at regular time intervals and the volume of the music 512 played is controlled in accordance with the applied function.

Further, the person may also use the volume control button 514 to select his setting with respect to the sound intensity of the music 512. In response to receiving the user setting, the controller adapts the applied function such that the function respects the user setting for the detected physiological state of the person at the moment of receiving the user setting. However, after adapting the function, the function is gradually changed towards the standard function. Thus, some time after providing the user setting, the alarm clock 510 controls the volume level according to the standard function.

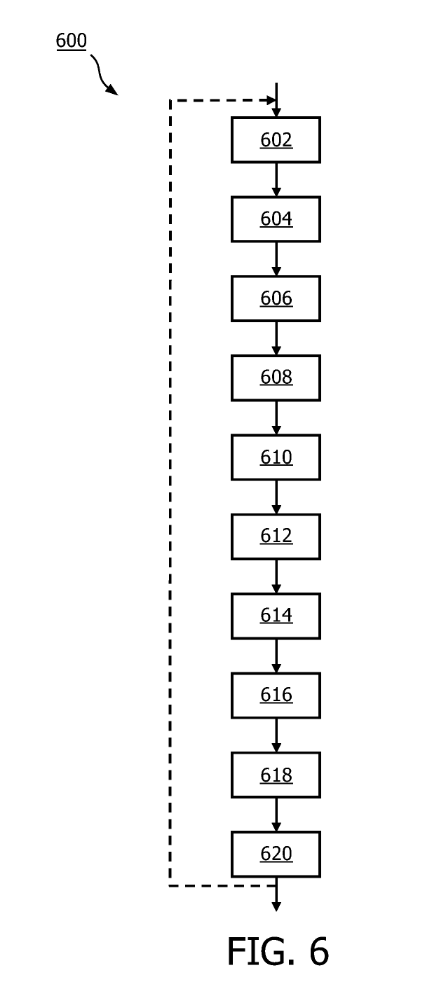

FIG. 6 shows a flow diagram of a method 600 of controlling an environmental physical characteristic in an environment of a person. In step 602 a sensor signal is obtained by a sensor. The sensor signal represents a physiological condition of a person. In step 604 the sensor signal is provided to a controller. Subsequently, a control signal is generated 606 by the controller by applying an original function to the sensor signal. The control signal indicates a desired influencing of the environmental physical characteristic. In step 608 the control signal is provided to an environment influencing means. Subsequently, the environment influencing means changes 610 the environmental physical characteristic in response to receiving the control signal. In step 612 a user setting signal is obtained by an input means. The user setting signal represents a user setting with respect to the environmental physical characteristic. In step 614 the user setting signal is provided to the controller. In response to receiving the user setting signal, the controller defines 616 an adapted function such that the adapted function provides the control signal which, if applied to the sensor signal occurring when the user setting signal was received, causes the environmental physical characteristic to become closer to the user setting than when the original function is applied to the sensor signal occurring when the user setting signal was received. In step 620 the adapted function is applied by the controller to generate the control signal. Subsequently, in step 620, the controller gradually changes the applied adapted function towards another function after the particular instant at which the user setting signal was received.

As shown in FIG. 6, the steps of the method may be executed in iterative loops. In a practical embodiment, steps 602 to 610 are executed in iterative loops while the steps 612 to 618 are executed in parallel with the execution of steps 602 to 610 when a user setting is provided by the user to the input means. Further, the step 620 of gradually changing may be performed during every loop of executing steps 602 to 610, or may be performed at regular time intervals in parallel with the execution of the steps 602 to 610.

It should be noted that the above-mentioned embodiments illustrate rather than limit the invention, and that those skilled in the art will be able to design many alternative embodiments without departing from the scope of the appended claims.

In the claims, any reference signs placed between parentheses shall not be construed as limiting the claim. Use of the verb “comprise” and its conjugations does not exclude the presence of elements or steps other than those stated in a claim. The article “a” or “an” preceding an element does not exclude the presence of a plurality of such elements. The invention may be implemented by means of hardware comprising several distinct elements, and by means of a suitably programmed computer. In the device claim enumerating several means, several of these means may be embodied by one and the same item of hardware. The mere fact that certain measures are recited in mutually different dependent claims does not indicate that a combination of these measures cannot be used to advantage.

1. A system for controlling an environmental physical characteristic in an environment of a person, comprising
an environment influencing means for changing the environmental physical characteristic in response to a control signal indicating a desired influencing of the environmental physical characteristic,
a sensor for obtaining a sensor signal representing a physiological condition of the person,
an input means for obtaining a user setting signal representing a user setting with respect to the environmental physical characteristic,
a controller for receiving the sensor signal and the user setting signal and for generating the control signal by applying an original function to the sensor signal,
wherein the controller is configured for:
i) defining an adapted function in response to receiving the user setting signal, wherein the adapted function provides the control signal which, if applied to the sensor signal occurring when the user setting signal was received, causes the environmental physical characteristic to become closer to the user setting than when the original function is applied to the sensor signal occurring when the user setting signal was received,
ii) applying the adapted function to generate the control signal,
iii) gradually changing the applied adapted function towards another function.

2. A system according to claim 1, wherein the adapted function provides the control signal which, if applied to the sensor signal occurring when the user setting was received, causes the environmental physical characteristic to become substantially equal to the user setting.

3. A system according to claim 1, wherein
the controller is configured for applying a previous function before the particular moment of receiving the user setting signal, and
the controller is configured for combining a further function with a limited bandwidth with the previous function to define the adapted function, wherein the limited bandwidth is a predefined range in which the further function provides a value substantially different from zero and wherein the limited bandwidth is arranged around the value of the sensor signal occurring when the user setting signal was received.

4. A system according to claim 3, wherein the further function is based on a Gaussian function.

5. A system according to claim 1, wherein the controller is further configured for defining the adapted function such that, if the sensor signal varies within a predefined variability interval, the control signal remains within a predefined deviation interval.

6. A system according to claim 1, wherein the controller is configured for starting the gradual change of the applied adapted function only after receiving the sensor signal having a value outside a predefined range around the value of the sensor signal occurring when the user setting signal was received.

7. A system according to claim 1, wherein
the controller is configured for storing a standard predefined function, and
the gradual change of the applied adapted function is towards the standard predefined function.

8. A system according to claim 1, wherein
the controller is configured for applying a previous function before the particular instant of receiving the user setting signal, and
the controller is further configured for defining, after defining the adapted function, said another function by combining the adapted function and the previous function.

9. A system according to claim 8, wherein said another function is a weighted average of the previous function and the adapted function.

10. A system according to claim 1, wherein the environmental physical characteristic comprises at least one of the following: characteristics of light of the environment, characteristics of sound in the environment, temperature, humidity, characteristics or movements of objects or parts of objects in the environment, characteristics or movements of objects with which the person is in physical contact, or a scent of the environment.

11. A system according to claim 1, wherein the sensor signal comprises information relating to at least one of the following physiological conditions of the person: skin temperature, core body temperature, skin conductivity, blood pressure, heart rate, heart rate variability, movements of the person, respiration frequency, respiration frequency variability, EEG activity, eye activity, characteristics of sound directly relating to the person or ECG information.

12-13. (canceled)

14. A method of controlling an environmental physical characteristic in an environment of a person, the method comprising the steps of:
obtaining a sensor signal by a sensor, the sensor signal representing a physiological condition of the person,
providing the sensor signal to a controller,
generating a control signal by the controller by applying an original function to the sensor signal, the control signal indicating a desired influencing of the environmental physical characteristic,
providing the control signal to an environment influencing means,
changing the environmental physical characteristic by the environmental influencing means in response to receiving the control signal,
obtaining a user setting signal by an input means, the user setting signal representing a user setting with respect to the environmental physical characteristic,
providing the user setting signal to the controller,
defining an adapted function providing the control signal which, if applied to the sensor signal occurring when the user setting was received, causes the environmental physical characteristic to become closer to the user setting than when the original function is applied to the sensor signal occurring when the user setting was received,
applying the adapted function in the controller, and
gradually changing the adapted function towards another function.

15. A computer program product comprising instructions for causing a processor system to perform the method according to claim 14.
